(19) United States
(12) Patent Application Publication
(10) Pub. No.: US 2011/0213467 A1
(43) Pub. Date: Sep. 1, 2011

(54) ORTHOPAEDIC IMPLANT WITH WOVEN INGROWTH MATERIAL

(75) Inventors: Antony J. Lozier, Warsaw, IN (US); Russell M. Parrott, Warsaw, IN (US); Dennis R. Aquilo, Youngsville, NC (US); John E. Meyers, Columbia City, IN (US)

(73) Assignee: ZIMMER, INC., Warsaw, IN (US)

(21) Appl. No.: 12/873,794

(22) Filed: Sep. 1, 2010

Related U.S. Application Data
(63) Continuation of application No. 12/690,295, filed on Jan. 20, 2010, now abandoned.
(60) Provisional application No. 61/145,821, filed on Jan. 20, 2009.

(51) Int. Cl. A61F 2/38; A61F 2/28

(52) U.S. Cl. 623/20.32; 623/16.11

(57) ABSTRACT

Methods and apparatuses for attaching soft tissue and bone to orthopaedic implants. The orthopaedic implants are provided with a woven ingrowth material to facilitate attachment of soft tissue and bone thereto. In one embodiment, the woven ingrowth material is formed as a patch or region of woven material attached to the implant. In another embodiment, a sleeve formed of woven ingrowth material encompasses the majority of the implant.

### CROSS-REFERENCE TO RELATED APPLICATION

This application is a continuation of U.S. patent application Ser. No. 12/690,295 entitled “Orthopaedic Implant with Woven Ingrowth Material,” filed Jan. 20, 2010, which claims priority from U.S. Provisional Patent Application No. 61/145,821, entitled “Orthopaedic Implant with Woven Ingrowth Material,” filed on Jan. 20, 2009, by the same inventors hereof, the disclosures of which are expressly incorporated herein by reference.

### BACKGROUND

1. Field of the Disclosure.

The present disclosure relates to methods and apparatuses for attaching tissue structures, including soft tissue structures and/or bone, to orthopaedic implants. More particularly, the present disclosure relates to methods and apparatuses for attaching soft tissue structures and/or bone to an orthopaedic implant using woven ingrowth material on the implant.

2. Description of the Related Art.

Orthopaedic implants are commonly used to replace at least a portion of a patient's joint in order to restore the use of the joint, or to increase the use of the joint, following deterioration due to aging or illness, injury due to trauma, or disease.

### SUMMARY

The present disclosure provides methods and apparatuses for attaching soft tissue and/or bone to orthopaedic implants. The orthopaedic implants are provided with a woven ingrowth material to facilitate attachment of soft tissue and/or bone thereto. In one embodiment, the woven ingrowth material is formed as a patch or region of woven material attached to the implant. In another embodiment, a sleeve formed of woven ingrowth material encompasses the majority of the implant.

In one form thereof, the present disclosure provides an orthopaedic implant assembly for positioning in a bone adjacent a tissue structure. The orthopaedic implant assembly includes an orthopaedic implant suitable for replacing at least a portion of the bone, a woven material having a first, body portion that is fixedly secured to said orthopaedic implant to prevent relative movement between said body portion of said woven material and said orthopedic implant, and a second, extension portion that extends freely away from said orthopaedic implant, and a fastener sized for securement into said woven material to operably secure the tissue structure to said orthopaedic implant. When the orthopaedic implant is positioned adjacent the tissue structure, the woven material is accessible outside the bone and the tissue structure is securable to the orthopaedic implant via attachment of the fastener into the woven material.

In another form thereof, the present disclosure provides an orthopaedic implant assembly for positioning in a tibia adjacent a tissue structure. The orthopaedic implant assembly includes a tibial implant suitable for replacing at least a portion of the tibia, a woven material, and a fastener. The tibial implant includes a body having a proximal end, a distal end opposite the proximal end, an anterior surface, a posterior surface opposite the anterior surface, a medial surface, and a lateral surface opposite the medial surface, and a tray coupled to the proximal end of the body. The woven material has a first, body portion that is fixedly secured to the tibial implant to prevent relative movement between the body portion of the woven material and the tibial implant, and a second, extension portion that extends freely away from the tibial implant. The fastener is sized for securement into said woven material to operably secure the tissue structure to said tibial implant. When the tibial implant is positioned adjacent the tissue structure, the woven material is accessible outside the tibia and the tissue structure is securable to the tibial implant via attachment of the fastener into the woven material.

In yet another form thereof, the present disclosure provides a method for attaching a tissue structure to an orthopaedic implant, the orthopaedic implant implantable in a bone. The method includes the steps of: providing an orthopaedic implant with a woven material secured thereto, the woven material having a first, body portion that is fixedly secured to the orthopaedic implant to prevent relative movement between the body portion of the woven material and the orthopedic implant, and a second, extension portion that extends freely away from the orthopaedic implant; positioning the orthopaedic implant in the bone such that the woven material is accessible outside the bone; and securing the tissue structure to the woven material by connecting a fastener directly into the woven material.

### BRIEF DESCRIPTION OF THE DRAWINGS

The above-mentioned and other features of the disclosure, and the manner of attaining them, will become more apparent and will be better understood by reference to the following description of embodiments of the disclosure taken in conjunction with the accompanying drawings, wherein:

FIG. 1 is a perspective view of an orthopaedic proximal tibial implant with a woven ingrowth material region on an anterior surface thereof and an extension portion formed of the woven ingrowth material extending from the implant;

FIG. 2 is a perspective view of another orthopaedic tibial implant with a woven ingrowth material region on a posterior surface thereof;

FIG. 3 is a perspective view of yet another orthopaedic tibial implant with a woven ingrowth material region on a lateral surface thereof;

FIG. 4 is a perspective view of an orthopaedic proximal tibial implant with a woven ingrowth material region on a medial surface thereof;

FIG. 5 is a perspective view of an orthopaedic proximal tibial implant with a sleeve disposed therearound and formed of a woven ingrowth material;

FIG. 6 is a perspective view of an orthopaedic proximal femoral implant with a plurality of woven ingrowth material regions; and

FIG. 7 is a perspective view of another orthopaedic proximal femoral implant with a plurality of woven ingrowth material regions.

Corresponding reference characters indicate corresponding parts throughout the several views. The exemplifications set out herein illustrate embodiments of the disclosure and such exemplifications are not to be construed as limiting the scope of the invention in any manner.

### DETAILED DESCRIPTION

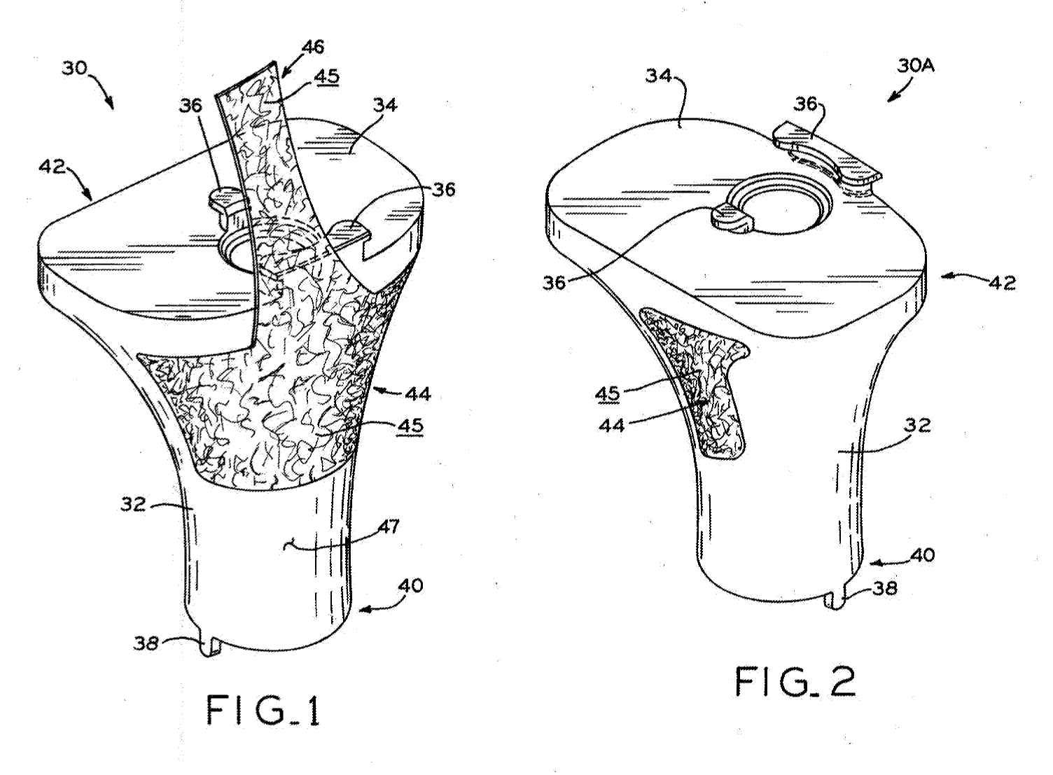

Referring now to FIG. 1, proximal tibial implant 30 is shown and may be used to restore mechanical and biological fixation of bone and/or soft tissue structures, such as muscle, ligaments, capsules, and/or tendons, for example, associated with a knee joint of a patient to enhance the stability of the knee joint and to restore knee joint kinematics. Proximal tibial implant 30 may be used in a patient requiring complete metaphyseal removal of the proximal tibia. Proximal tibial implant 30 may include body 32 having proximal end 42 and distal end 40, tibial tray or plate 34, and mating structure 36.

Body 32 may be formed from relatively light-weight material, such as titanium, a cobalt chromium alloy, or other suitable biocompatible material, for example, thereby making it easier for the patient to lift and extend the knee joint, particularly in procedures which require extensive removal of muscle proximate the knee joint. In one embodiment, tibial plate 34 may be formed as a modular component of proximal tibial implant 30 to provide more interoperative options. In an exemplary embodiment, tibial plate 34 is formed as a wear-resistant tibial plate to minimize debris from articulation with another component (not shown) of the knee joint. Mating structure 36 may be formed complementary to a meniscal component (not shown) of the knee joint to provide either a mobile or a non-mobile bearing connection between proximal tibial implant 30 and the meniscal component. Proximal tibial implant 30 may also include rotational adjustment tabs 38 at distal end 40 to allow for in vivo rotational adjustment of proximal tibial implant 30 relative to another implant or to the remaining structure of the tibia. Rotational adjustment tabs 38 may generally extend distally from body 32 along a lateral and/or a medial side of implant 30. A plurality of tabs 38 or, alternatively, a single tab 38 may be utilized.

In an exemplary embodiment, proximal tibial implant 30 also includes at least one bone and/or soft tissue attachment region 44. Attachment region 44 may be positioned on an anterior surface of proximal tibial implant 30 and may be integrally formed with proximal tibial implant 30. In one embodiment, attachment region 44 is formed as a modular component of proximal tibial implant 30. Attachment region 44 may be positioned in a recess provided on the anterior surface of proximal tibial implant 30. Attachment region 44 may generally have a relatively thin thickness, e.g., as low as approximately 1 mm to as high as approximately 5 mm, such as to define a relatively slim profile such that attachment region 44 does not protrude from proximal tibial implant 30 and consequently potentially interfere with other anatomical structures. For example, in an exemplary embodiment, the anterior surface of attachment region 44 is substantially flush with anterior surface 47 of proximal tibial implant 30. Attachment region 44 provides a direct connection between bone and/or a soft tissue structure and proximal tibial implant 30. For example, a patella tendon, which joins a lower edge of a patella (not shown) of the knee joint with a tibial tubercle of a tibia, may be directly attached via mechanical and/or biological fixation to proximal tibial implant 30 after implantation of implant 30 via attachment region 44. Such fixation of the patella tendon to proximal tibial implant 30 enhances usability of the knee joint. For example, when a patient jumps into the air or allows the tibia to hang without any support, the fixation of the patella tendon to proximal tibial implant 30 prevents dislocation of the components of the prosthetic knee joint and facilitates normal functioning of the prosthetic knee joint after the jump or once the tibia is again supported. In one example, a rotating hinged knee includes a femoral component with a post extending through a meniscal component and into a tibial component. During a jump or when the tibia is unsupported, the patella tendon effectively prevents the post from extending too far from the tibial component and ensures that the post returns to proper engagement with the tibial component once normal functioning is resumed. As described further below, attachment region 44 may include at least one porous surface 45, such as a surface to facilitate ingrowth of bone and/or soft tissues.

In an exemplary embodiment, attachment region 44 is formed from a woven ingrowth material which facilitates ingrowth of soft tissue, bone, and/or other anatomical structures. For the purposes of this document, the term “woven” is defined as a material made or constructed by interlacing threads, strips, or pieces of material or other elements into a whole. Unlike an embroidered material that is formed by stitching and knotting threads into a pre-existing fabric, a woven material provides more control over the materials used the construct the final product and the mechanical properties of the final product. The woven ingrowth material may be formed from various materials, including, but not limited to, metals (i.e., titanium, tantalum, and cobalt chromium alloys), synthetic polymers (i.e., ultra-high molecular weight polyethylene (UHMWPE), polyether-ether-ketone (PEEK), and polyether-ketone (PEK)), bioresorbable fibers (i.e., PLA, PGA, PLLA, and PU), ceramics/glass (i.e., alumina, zirconia, and pyrolytic carbon), biological material (i.e., collagen, silk, and chitosan), and/or a composite of the same (i.e., carbon reinforced PEEK). The woven ingrowth material may be a flexible material to facilitate conforming attachment region 44 to a surface of an implant. In one exemplary embodiment, attachment region 44 may have a Young's modulus that is substantially similar to the Young's modulus of the surrounding bone, which may range from 12 to 20 GPa. The woven ingrowth material may have variable pore sizes such that the pore sizes may be denser near areas of contact with the implant to facilitate attachment of attachment region 44 to the implant. The woven ingrowth material may have a variable thickness and/or variable roughness or variation in pattern. The woven ingrowth material of attachment region 44 may be affixed to implant 30 via a fastener or an attachment mechanism such as, but not limited to, an adhesive such as a bioadhesive, diffusion bonding, a hook-and-loop fastener, a screw, a nail, a bolt, an integral fit, an interference fit, or attachment region 44 may be integrally formed with implant 30.

The woven ingrowth material may be configured to allow bone, soft tissue, and blood vessels to grow through attachment region 44, thereby reducing the potential of subcutaneous irritation and necrosis of the soft tissue structure. The woven ingrowth material may include tissue ingrowth enhancement properties, such as bone or tissue growth factors. The woven ingrowth material may also include other pharmacological and/or therapeutic agents. The woven ingrowth material may include seams therein to enhance the strength of the material and these seams may be created via laser welding, ultrasonic sealing, stitching, or other suitable methods. Examples of woven material which may be used with the present disclosure include the Artelon® CMC Spacer, available from Small Bone Innovations, Inc. of Morrisville, Pa., which has material properties including ultimate stress greater than 240 MPa and a porosity of approximately 90%; and the LARS™ Ligament Augmentation and Reconstruction System available from Corin Group PLC of Gloucestershire, United Kingdom.

In one embodiment, the woven ingrowth material may be a three-dimensional woven material as described in U.S. patent application Ser. No. 12/131,188 entitled “Implant Sensors” and/or U.S. patent application Ser. No. 12/640,655 entitled “In-Line Treatment of Yarn Prior to Creating a Fabric,” the disclosures of which are expressly incorporated by reference herein.

In another embodiment, the woven ingrowth material may be formed as a material having a cellular structure which resembles bone and approximates the physical and mechanical properties of bone, thereby enabling rapid and extensive bone and/or soft tissue infiltration and strong attachment of bone and/or soft tissue structures thereto. For example, the material may be a highly porous biomaterial having a porosity as low as 55, 65, or 75 percent and as high as 80, 85, or 90 percent. The woven material may be formed to mimic material produced using Trabecular Metal™ technology generally available from Zimmer, Inc., of Warsaw, Indiana. Trabecular Metal™ is a trademark of Zimmer Technology, Inc. Such a woven material may be formed with a similar process as that described by a chemical vapor deposition (“CVD”) process in the manner disclosed in detail in U.S. Pat. No. 5,282,861, the disclosure of which is expressly incorporated herein by reference. The woven material may include up to 75%-85% or more void space therein. The woven material may be made in a variety of densities in order to selectively tailor the structure for particular applications.

Implant 30 may also include extension region 46 formed of the same material and structure as attachment region 44 and having porous surface 45. In one exemplary embodiment, extension region 46 may have a Young's modulus that is substantially similar to the Young's modulus of the patella tendon, which may range from 1.3 to 2.2 GPa. In an exemplary embodiment, extension region 46 and attachment region 44 are formed as a unitary construct. In another embodiment, extension region 46 is attached to attachment region 44 via sutures, adhesive, or other suitable connection, either before or after affixation of attachment region 44 to implant 30.

In operation, a surgeon may attach a soft tissue structure, such as muscle, a ligament, a capsule, and/or a tendon, for example, and/or bone to proximal tibial implant 30. The surgeon positions the soft tissue structure and/or bone in contact with the anterior surface of attachment region 44. At least one suture may be threaded through the woven ingrowth material of attachment region 44 at any desired location on attachment region 44 to attach the soft tissue structure and/or bone at least temporarily to implant 30. In contrast to sutures, a surgeon may also use surgical tape or surgical cables, for example. In one embodiment, the sutures may be biodegradable after a period of time in which the soft tissue structure and/or bone is permanently attached to proximal tibial implant 30 via ingrowth of the soft tissue structure and/or bone into attachment region 44. The ability to suture or fasten the soft tissue structure and/or bone directly to the woven ingrowth material and to suture therethrough eliminates the need for any apertures proximate attachment region 44 and extending through implant 30 for threading sutures therethrough. The potential risk of suture breakage is thereby substantially eliminated because the woven ingrowth material has no potential sharp edges. In this manner, the soft tissue structure and/or bone may be both biologically and mechanically affixed to proximal tibial implant 30. The close proximity of the suture attachments in attachment region 44 in relation to the soft tissue structure and/or bone further facilitates more direct contact between the soft tissue structure and/or bone and attachment region 44. Alternatively, the soft tissue structure and/or bone may be first attached to attachment region 44 and then attachment region 44 is attached to body 32, thereby providing potential for tensioning the soft tissue structure and/or bone prior to securement of attachment region 44 to body 32. In yet another embodiment, the soft tissue structure and/or bone is positioned between attachment region 44 and body 32 and then attachment region 44 is attached to body 32.

In an exemplary procedure, the surgeon may attach the soft tissue structure and/or bone to implant 30 via extension region 46, thereby providing an extension for attaching the soft tissue structure and/or bone. This may be required in situations in which the soft tissue structure and/or bone does not extend completely across implant 30 into contact with attachment region 44. The surgeon may use a similar procedure to affix the soft tissue structure and/or bone to extension region 46 as used to affix the same to attachment region 44, as described above.

Referring now to FIG. 2, an alternative embodiment proximal tibial implant 30A is shown and is substantially identical to proximal tibial implant 30, described above with reference to FIG. 1, except as described below. Proximal tibial implant 30A includes body 32, tibial plate 34, mating structure 36, rotational adjustment tabs 38, distal end 40, proximal end 42, and at least one attachment region 44. Proximal tibial implant 30A differs from proximal tibial implant 30 (FIG. 1) in that attachment region 44 is provided on a posterior side of proximal tibial implant 30A. Proximal tibial implant 30A may also include an extension region similar to extension region 46 (FIG. 1). For example, proximal tibial implant 30A may include a woven material extending proximally from attachment region 44 on the posterior side of proximal tibial implant 30A.

In operation, a surgeon may attach a soft tissue structure, such as a muscle, a ligament, a capsule, and/or a tendon, for example, and/or bone to proximal tibial implant 30A. The surgeon positions the soft tissue structure and/or bone in contact with the surface of attachment region 44 and secures the soft tissue structure and/or bone in a substantially identical manner as described above with respect to proximal tibial implant 30 (FIG. 1).

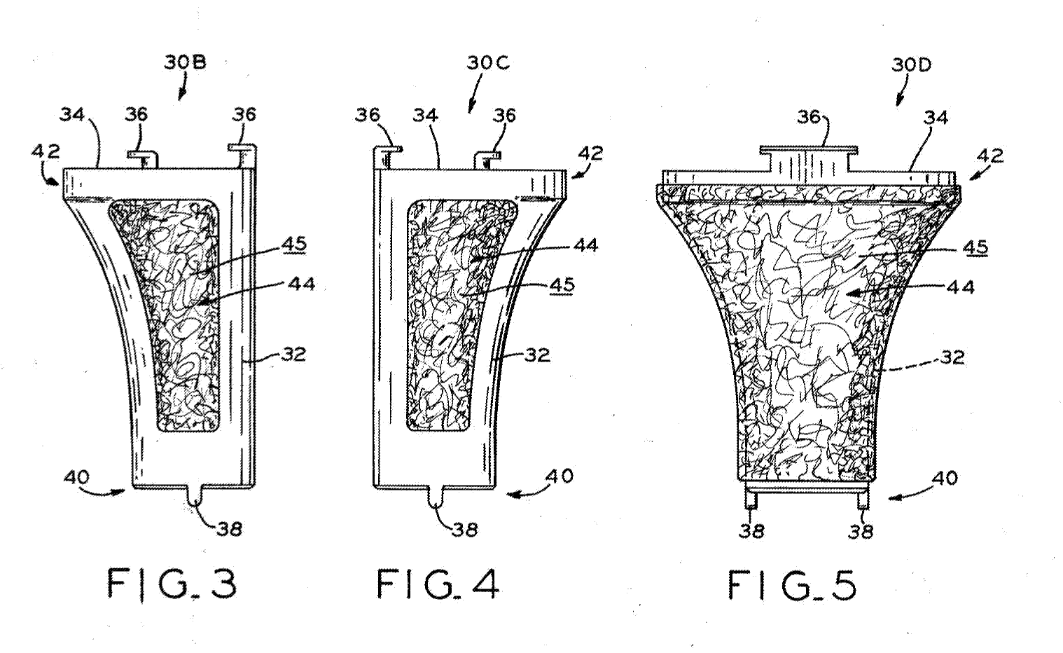

Referring now to FIG. 3, an alternative embodiment proximal tibial implant 30B is shown and is substantially identical to proximal tibial implant 30, described above with reference to FIG. 1, except as described below. Proximal tibial implant 30B includes body 32, tibial plate 34, mating structure 36, rotational adjustment tabs 38, distal end 40, proximal end 42, and at least one attachment region 44. Proximal tibial implant 30B is designed for a right knee and differs from proximal tibial implant 30 (FIG. 1) in that attachment region 44 is provided on a lateral side of proximal tibial implant 30B instead of an anterior side of the implant. Proximal tibial implant 30B may also include an extension region similar to extension region 46 (FIG. 1). For example, proximal tibial implant 30B may include a woven material extending proximally from attachment region 44 on the lateral side of proximal tibial implant 30B.

In operation, a surgeon may attach a soft tissue structure, such as a muscle, a ligament, a capsule, and/or a tendon, for example, and/or bone to proximal tibial implant 30B. The surgeon positions the soft tissue structure and/or bone in contact with the surface of attachment region 44 and secures the soft tissue structure and/or bone in a substantially identical manner as described above with respect to proximal tibial implant 30 (FIG. 1).

Referring now to FIG. 4, an alternative embodiment proximal tibial implant 30C is shown and is substantially identical to proximal tibial implant 30, described above with reference to FIG. 1, except as described below. Proximal tibial implant 30C includes body 32, tibial plate 34, mating structure 36, rotational adjustment tabs 38, distal end 40, proximal end 42, and at least one attachment region 44. Proximal tibial implant 30C is designed for a right knee and differs from proximal tibial implant 30 (FIG. 1) in that attachment region 44 is provided on a medial side of proximal tibial implant 30C instead of an anterior side of the implant. Proximal tibial implant 30C may also include an extension region similar to extension region 46 (FIG. 1). For example, proximal tibial implant 30C may include a woven material extending proximally from attachment region 44 on the medial side of proximal tibial implant 30C.

In operation, a surgeon may attach a soft tissue structure, such as a muscle, a ligament, a capsule, and/or a tendon, for example, and/or bone to proximal tibial implant 30C. The surgeon positions the soft tissue structure and/or bone in contact with the surface of attachment region 44 and secures the soft tissue structure and/or bone in a substantially identical manner as described above with respect to proximal tibial implant 30 (FIG. 1).

Although shown in FIGS. 1-4 as being exclusively positioned on either an anterior, a posterior, a lateral, or a medial side of a proximal tibial implant, attachment regions 44 may be positioned on the proximal tibial implant at any desired location upon which a surgeon desires to attach a soft tissue structure and/or bone to the proximal tibial implant. For example, attachment regions 44 may be positioned on the anterior, lateral, and medial sides, with no attachment region 44 on the posterior side. Moreover, attachment regions 44 may be positioned at intermediate locations on the proximal tibial implant, such as lateral-anterior or medial-posterior locations, for example, or at any other locations advantageous for securing bone and/or soft tissue thereto.

Referring now to FIG. 5, an alternative embodiment proximal tibial implant 30D is shown and is substantially identical to proximal tibial implant 30, described above with reference to FIG. 1, except as described below. Proximal tibial implant 30D includes body 32, tibial plate 34, mating structure 36, rotational adjustment tabs 38, distal end 40, proximal end 42, and attachment region 44. Proximal tibial implant 30D differs from proximal tibial implant 30 (FIG. 1) in that attachment region 44 is provided as a sleeve disposed on proximal tibial implant 30D. The sleeve of woven ingrowth material provided by attachment region 44 may be affixed to proximal tibial implant 30D via any of the attachment mechanisms described above. The sleeve may be attached during a surgical procedure after the surgeon has determined that the attachment of a soft tissue structure and/or bone is needed. This can be done by selecting a prepackaged and sterile sleeve that matches the desired implant. The sleeve can be attached using hook and loop fasteners, tying fasteners, adhesives, and/or mechanical fasteners, such as screws, for example. The sleeve may also be assembled preoperatively by the manufacturer.

In operation, a surgeon may attach a soft tissue structure, such as a muscle, a ligament, a capsule, and/or a tendon, for example, and/or bone to proximal tibial implant 30D. The surgeon positions the soft tissue structure and/or bone in contact with the surface of attachment region 44 and secures the soft tissue structure in a substantially identical manner as described above with respect to proximal tibial implant 30 (FIG. 1). The sleeve construct of attachment region 44 allows a surgeon to select any location on proximal tibial implant 30D at which to attach the soft tissue structure and/or bone without being limited to a specific region of proximal tibial implant 30D.

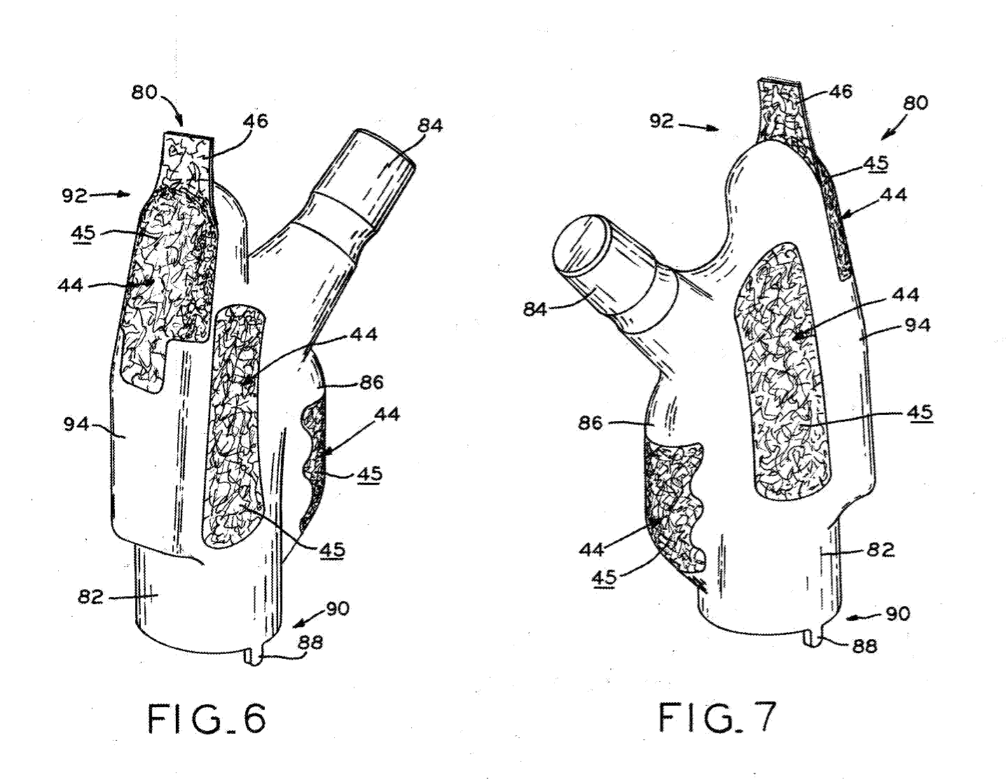

Referring now to FIGS. 6 and 7, proximal femoral implant 80 is shown and may be used to restore mechanical and biological fixation of soft tissue and/or bone associated with a hip joint of a patient to enhance the stability of the hip joint, to restore hip joint function, and to enhance hip joint kinematics. Proximal femoral implant 80 may typically be used in a patient requiring complete metaphyseal removal of the proximal femur. Proximal femoral implant 80 may include body 82 including proximal end 92 and distal end 90. Proximal femoral implant 80 may also include neck 84 configured to mate with a corresponding femoral head component (not shown) of a prosthetic hip joint. The femoral head component may articulate against a natural or a prosthetic acetabulum. Proximal femoral implant 80 may also include anteversion adjustment tabs 88 at distal end 90 to allow for in vivo rotational adjustment of proximal femoral implant 80 relative to a prosthetic femoral stem component or the natural femur. Anteversion adjustment tabs 88 may generally extend distally from body 82 along a lateral and/or a medial side of implant 80. A plurality of tabs 88 or, alternatively, a single tab 88 may be utilized.

Proximal femoral implant 80 may include medial protrusion 86 and lateral protrusion 94. Medial protrusion 86 may include at least one attachment region 44. Medial protrusion 86 may buttress neck 84 and provide additional support to proximal femoral implant 80 when implanted into a hip joint. Lateral protrusion 94 may also include at least one attachment region 44. As shown in FIGS. 6 and 7, attachment region 44 on lateral protrusion 94 of proximal femoral implant 80 includes extension region 46 that extends proximally from proximal femoral implant 80 to facilitate attachment of soft tissue and/or bone thereto. Attachment regions 44 may also be provided on an anterior surface or a posterior surface of proximal femoral implant 80. Moreover, attachment regions 44 may be provided at any locations on proximal femoral implant 80 advantageous for securing bone and/or soft tissue thereto.

In operation, a surgeon may attach a plurality of soft tissue structures, such as a muscle, a ligament, a capsule, and/or a tendon, for example, and/or bone to proximal femoral implant 80. The surgeon positions the soft tissue structure and/or bone in contact with the surface of any or all of attachment regions 44 and/or extension region 46 and secures the soft tissue structure and/or bone in a substantially identical manner as described above with respect to proximal tibial implant 30 (FIG. 1). In an exemplary embodiment, the soft tissue structure is an iliopsoas muscle which, when attached to proximal femoral implant 80, enhances stability of the hip joint. In another exemplary embodiment, the soft tissue structure is an abductor or quadriceps muscle which, when attached to proximal femoral implant 80, enhance the kinematics of the hip joint. In one embodiment, the attachment of the soft tissue structures to proximal femoral implant 80 provides a sling around the hip joint to maximize hip joint function and kinematics.

Although not illustrated in FIGS. 6 and 7, proximal femoral implant 80 may also include attachment region 44 formed as a sleeve, similar to that shown in FIG. 5, to provide an unlimited number of attachment points for a soft tissue structure and/or bone anywhere on proximal femoral implant 80.

Although the methods and apparatuses described in this disclosure describe attachment of natural soft tissue structures to orthopaedic implants, the methods and apparatuses may also be used to secure artificial soft tissue structures to orthopaedic implants in substantially similar manners.

Furthermore, although the methods and apparatuses described in this disclosure describe attachment of soft tissue and/or bone structures to segmental orthopaedic implants, the methods and apparatuses may also be used to secure soft tissue and/or bone structures to any orthopaedic implant, such as hip implants, knee implants, elbow implants, shoulder implants such as a reverse shoulder implant, extremity implants, and trauma implants.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An orthopaedic implant assembly for positioning in a bone adjacent a tissue structure, comprising:
an orthopaedic implant suitable for replacing at least a portion of the bone;
a woven material having a first, body portion that is fixedly secured to said orthopaedic implant to prevent relative movement between said body portion of said woven material and said orthopedic implant, and a second, extension portion that extends freely away from said orthopaedic implant; and
a fastener sized for securement into said woven material to operably secure the tissue structure to said orthopaedic implant;
whereby when said orthopaedic implant is positioned adjacent the tissue structure, said woven material is accessible outside the bone and the tissue structure is securable to said orthopaedic implant via attachment of said fastener into said woven material.

2. The orthopaedic implant assembly of claim 1, wherein said body portion of said woven material forms an attachment region on said orthopaedic implant, said attachment region positioned proximate at least one of an anterior surface, a posterior surface, a medial surface, a lateral surface, a proximal end, and a distal end of said orthopaedic implant.

3. The orthopaedic implant assembly of claim 1, wherein said woven material forms a sleeve, said sleeve disposed around said orthopaedic implant.

4. The orthopaedic implant assembly of claim 1, wherein said body portion of said woven material is integrally formed with said orthopaedic implant.

5. The orthopaedic implant assembly of claim 1, wherein said orthopaedic implant includes a recess sized to receive said body portion of said woven material.

6. The orthopaedic implant assembly of claim 1, wherein said extension portion of said woven material is more elastic than said body portion of said woven material.

7. The orthopaedic implant assembly of claim 6, wherein said body portion of said woven material has a modulus of elasticity greater than 5 GPa.

8. The orthopaedic implant assembly of claim 7, wherein said body portion of said woven material has a modulus of elasticity between approximately 12 and 20 GPa.

9. The orthopaedic implant assembly of claim 6, wherein said extension portion of said woven material has a modulus of elasticity less than 5 GPa.

10. The orthopaedic implant assembly of claim 9, wherein said extension portion of said woven material has a modulus of elasticity between approximately 1.3 and 2.2 GPa.

11. The orthopaedic implant assembly of claim 1, wherein said woven material includes a porosity gradient, said body portion of said woven material having a low porosity/high density region that contacts said orthopaedic implant and a high porosity/low density region that contacts the tissue structure.

12. An orthopaedic implant assembly for positioning in a tibia adjacent a tissue structure, comprising:
a tibial implant suitable for replacing at least a portion of the tibia, said tibial implant comprising:
a body having a proximal end, a distal end opposite said proximal end, an anterior surface, a posterior surface opposite said anterior surface, a medial surface, and a lateral surface opposite said medial surface; and
a tray coupled to said proximal end of said body;
a woven material having a first, body portion that is fixedly secured to said tibial implant to prevent relative movement between said body portion of said woven material and said tibial implant, and a second, extension portion that extends freely away from said tibial implant; and
a fastener sized for securement into said woven material to operably secure the tissue structure to said tibial implant;
whereby when said tibial implant is positioned adjacent the tissue structure, said woven material is accessible outside the tibia and the tissue structure is securable to said tibial implant via attachment of said fastener into said woven material.

13. The orthopaedic implant assembly of claim 12, wherein said body portion of said woven material forms an attachment region on said tibial implant, said attachment region positioned proximate at least said anterior surface of said tibial implant.

14. The orthopaedic implant assembly of claim 12, wherein said extension portion of said woven material extends proximally beyond said tray of said tibial implant.

15. The orthopaedic implant assembly of claim 12, wherein said body portion of said woven material is integrally formed with said tibial implant.

16. The orthopaedic implant assembly of claim 12, wherein said tibial implant includes a recess sized to receive said body portion of said woven material.

17. The orthopaedic implant assembly of claim 12, wherein said body portion of said woven material includes a first surface that contacts at least one of the anterior surface, the posterior surface, the medial surface, and the lateral surface of the tibial implant, and a second surface opposite the first surface that remains exposed to contact the tissue structure.

18. The orthopaedic implant assembly of claim 12, wherein said extension portion of said woven material is more elastic than said body portion of said woven material.

19. A method for attaching a tissue structure to an orthopaedic implant, the orthopaedic implant implantable in a bone, the method comprising the steps of:
providing an orthopaedic implant with a woven material secured thereto, said woven material having a first, body portion that is fixedly secured to said orthopaedic implant to prevent relative movement between said body portion of said woven material and said orthopedic implant, and a second, extension portion that extends freely away from said orthopaedic implant;
positioning said orthopaedic implant in the bone such that said woven material is accessible outside the bone; and
securing the tissue structure to said woven material by connecting a fastener directly into said woven material.

20. The method of claim 19, wherein said securing step comprises threading at least one suture directly into said woven material.

21. The method of claim 19, wherein said securing step comprises securing bone to said body portion of said woven material and securing a soft tissue structure to said extension portion of said woven material.

22. The method of claim 19, wherein said providing step comprises providing a proximal tibial implant, and said securing step comprises securing a patella tendon to said woven material.

23. The method of claim 19, wherein said providing step comprises providing a proximal femoral implant, and said securing step comprises securing at least one of an iliopsoas muscle, an abductor, and a quadriceps muscle to said woven material.
